(19) United States
(12) Patent Application Publication
(10) Pub. No.: US 2007/0249930 A1
(43) Pub. Date: Oct. 25, 2007

(54) METHOD AND SYSTEM FOR TRACKING DEVICES WITH MULTIPLE RF TRANSMIT CHANNELS USING MRI

(75) Inventor: YUDONG ZHU, CLIFTON PARK, NY (US)

Correspondence Address: GENERAL ELECTRIC COMPANY;GLOBAL RESEARCH, PATENT DOCKET RM. BLDG. K1-4A59, NISKAYUNA, NY, 12309, US

(73) Assignee: GENERAL ELECTRIC COMPANY, SCHENECTADY, NY (US)

(21) Appl. No.: 11/564,402

(22) Filed: Nov. 29, 2006

Related U.S. Application Data
(60) Provisional application No. 60/794,365, filed on Apr. 24, 2006.

(51) Int. Cl. A61B 5/05

(52) U.S. Cl. 600/411

(57) ABSTRACT

A method and system for monitoring the placement of a catheter during an interventional procedure is provided. The method integrates transmit coils with the catheter and controls the coils' RF transmission through additional channels that operate synchronously with a scanner's standard transmit channel. The method enables simultaneous planar roadmap imaging and device tracking, and automatically produces images showing device location as a projection onto the roadmap frames.

### CROSS REFERENCE TO RELATED APPLICATIONS

This application is related to Provisional Application U.S. Ser. No. 60/794,365, entitled “METHOD FOR TRACKING DEVICES WITH MULTIPLE RF TRANSIT CHANNELS”, filed Apr. 24, 2006, the contents of which are herein incorporated by reference and the benefit of priority to which is claimed under 35 U.S.C. 119(e).

### BACKGROUND OF THE INVENTION

This invention relates generally to magnetic resonance imaging (MRI), and more particularly, to device tracking in conjunction with and simultaneous to MRI.

A variety of MRI-based methods have been developed to monitor the placement of a catheter, probe or other invasive devices during an interventional procedure. In one current approach, small sensing coils are attached to the catheter at strategic sites and catheter location is inferred by collecting and processing MR signals picked up by the sensing coils. With another current approach, an MR catheter probe is employed for repeated transmit and receive, and the MR images thus produced reveal probe location. In applying either approach, separate acquisition of roadmap images is performed every once in a while to provide anatomical information and/or to assist navigation.

MRI-based device tracking represented by the ones described above may potentially replace x-ray fluoroscopy in some interventional procedures. At present however, such MRI-based approaches yet need to further improve in imaging speed and/or quality. For the examples described above, which interleave roadmap scans with tracking scans, an increase in update rate with one type of scans typically implies a decrease in update rate with the other. The fact that catheter location information and roadmap images are obtained separately also implies potential registration errors due to the lapse of time between the scans, as well as an overhead associated with extra steps of spatial-information processing/integration.

There is therefore a need for a MRI method that enables simultaneous planar roadmap imaging and device tracking.

### BRIEF DESCRIPTION OF THE INVENTION

In a first aspect, a MRI method that enables simultaneous planar roadmap imaging and device tracking is provided. It employs multiple RF transmit channels to enable simultaneity and produces images showing device location as a projection onto the roadmap frames. The method may be advantageously applied in cases where soft tissue near the device undergoes motion/deformation and frequent roadmap image-update is desired.

In a second aspect, a system for acquiring images during an interventional procedure using a multiple channel Magnetic Resonance Imaging (MRI) device is provided. The system comprises a plurality of channels for transmitting and/or receiving radiofrequency (RF) signals during a MRI imaging session, an interventional device comprising at least one auxiliary transmit coil, and, a pulse sequence generator for generating pulse sequences adapted to acquire at least one planar image of a region of interest and at least one image of an interventional device projected onto the planar image substantially simultaneously.

### BRIEF DESCRIPTION OF THE DRAWINGS

The features and advantages of the present invention will become apparent from the following detailed description of the invention when read with the accompanying drawings in which:

FIG. 1 is an illustration of an exemplary MRI system to which embodiments of the present invention are applicable;

FIG. 2 is a simplified illustration of a catheter device with integrated RF transmit coils to which embodiments of the present invention are applicable; and,

FIG. 3 is a simplified drawing of a representative parallel excitation pulse to which embodiments of the present invention are applicable.

### DETAILED DESCRIPTION OF THE INVENTION

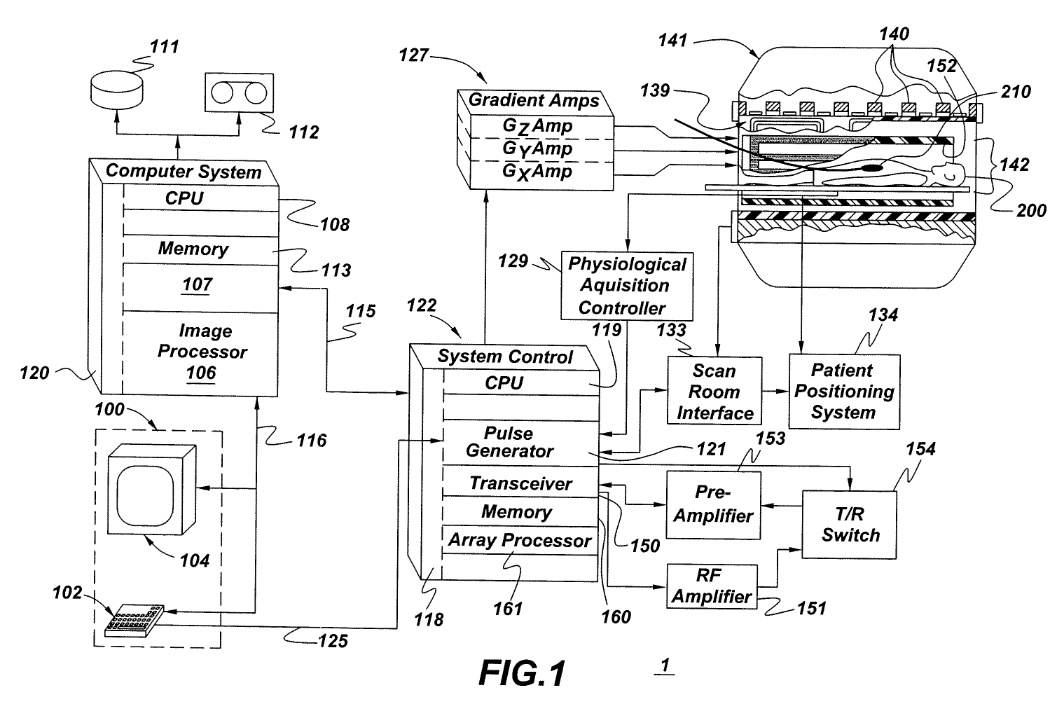

FIG. 1 illustrates a simplified block diagram of a system for producing images in accordance with embodiments of the present invention. In an embodiment, the system is an MR imaging system that incorporates embodiments of the present invention. The MR system could be, for example, a GE-Signa MR scanner available from GE Healthcare, which is adapted to perform the method of the present invention, although other systems could be used as well.

The operation of the MR system is controlled from an operator console 100 which includes a keyboard and control panel 102 and a display 104. The console 100 communicates through a link 116 with a separate computer system 107 that enables an operator to control the production and display of images on the screen 104. The computer system 107 includes a number of modules which communicate with each other through a backplane. These include an image processor module 106, a CPU module 108, and a memory module 113, known in the art as a frame buffer for storing image data arrays. The computer system 107 is linked to a disk storage 111 and a tape drive 112 for storage of image data and programs, and it communicates with a separate system control 122 through a high speed serial link 115.

The system control 122 includes a set of modules connected together by a backplane. These include a CPU module 119 and a pulse generator module 121 that connects to the operator console 100 through a serial link 125. It is through this link 125 that the system control 122 receives commands from the operator that indicates the scan sequence that is to be performed. The pulse generator module 121 operates the system components to carry out the desired scan sequence. It produces data that indicate the timing, strength, and shape of the radio frequency (RF) pulses which are to be produced, and the timing of and length of the data acquisition window. The pulse generator module 121 connects to a set of gradient amplifiers 127, to indicate the timing and shape of the gradient pulses to be produced during the scan. The pulse generator module 121 also receives subject data from a physiological acquisition controller 129 that receives signals from a number of different sensors connected to the subject 200, such as ECG signals from electrodes or respiratory signals from a bellows. And finally, the pulse generator module 121 connects to a scan room interface circuit 133 that receives signals from various sensors associated with the condition of the subject 200 and the magnet system. It is also through the scan room interface circuit 133 that a positioning device 134 receives commands to move the subject 200 to the desired position for the scan.

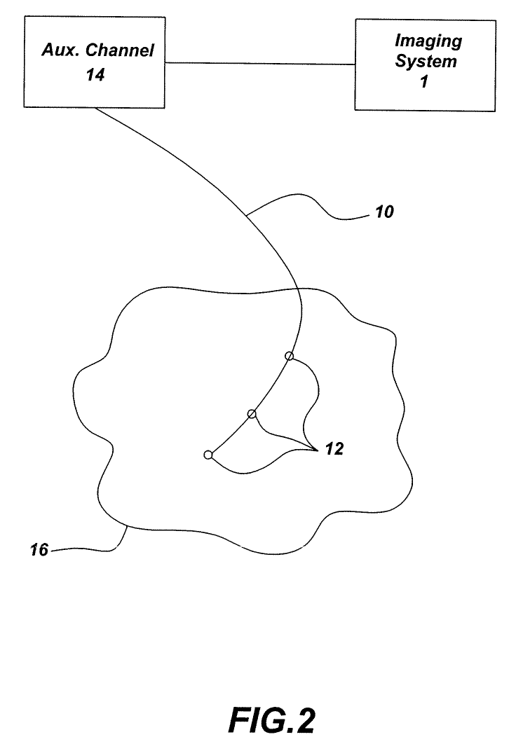

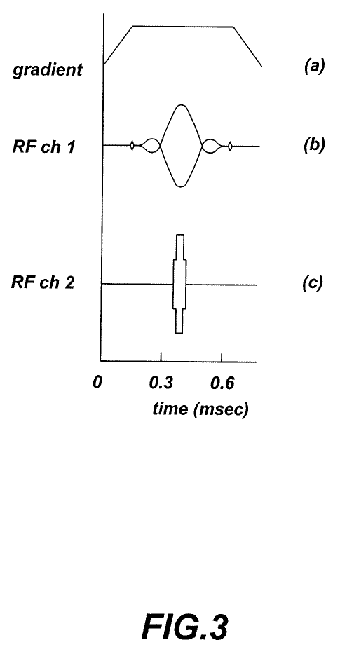

The gradient waveforms produced by the pulse generator module 121 are applied to a gradient amplifier system 127 comprised of Gₓ, G_{y} and G_{z} amplifiers. Each gradient amplifier excites a corresponding gradient coil in an assembly generally designated 139 to produce the magnetic field gradients used for position encoding acquired signals. The gradient coil assembly 139 forms part of a magnet assembly 141 which includes a polarizing magnet 140 and a RF coil system 152. Volume 142 is shown as the area within magnet assembly 141 for receiving subject 200 and includes a patient bore. As used herein, the usable volume of a MRI scanner is defined generally as the volume within volume 142 that is a contiguous area inside the patient bore where homogeneity of main, gradient and RF fields are within known, acceptable ranges for imaging. A transceiver module 150 in the system control 122 produces pulses that are amplified by a RF amplifier system 151 and coupled to the RF coil system 152 by a transmit/receive switch system 154. The resulting signals radiated by the excited nuclei in the subject 200 may be sensed by the same RF coil system 152 and coupled through the transmit/receive switch system 154 to a preamplifier system 153. The amplified MR signals are demodulated, filtered, and digitized in the receiver section of the transceiver 150. The transmit/receive switch 154 is controlled by a signal from the pulse generator module 121 to electrically connect the RF amplifier system 151 to the RF coil system 152 during the transmit mode (i.e., during excitation) and to connect the preamplifier system 153 during the receive mode. The transmit/receive switch system 154 also enables a separate RF coil, for example, a head coil or surface coil to be used in either the transmit or receive mode. In embodiments of the present invention, embodiments of the separate RF coil will be described with reference to FIGS. 2 and 3. During the transmit mode, the RF pulse waveforms produced by the pulse generator module 121 are applied to a RF amplifier system 151 comprised of multiple amplifiers. Each amplifier controls the current in a corresponding component coil of the RF coil system 152 in accordance with the amplifier's input RF pulse waveform. With the transmit/receive switch system 154, the RF coil system 152 is configured to perform transmission only, or alternatively, configured to additionally act as a receive coil array during receive mode. As used herein, “adapted to”, “configured” and the like refer to mechanical or structural connections between elements to allow the elements to cooperate to provide a described effect; these terms also refer to operation capabilities of electrical elements such as analog or digital computers or application specific devices (such as an application specific integrated circuit (ASIC)) that is programmed to perform a sequel to provide an output in response to given input signals.

The MR signals picked up by the RF coil system 152 or a separate receive coil (not shown, for example, a body, head, extremity or surface coil) are digitized by the transceiver module 150 and transferred to a memory module 160 in the system control 122. When the scan is completed and an entire array of data has been acquired in the memory module 160, an array processor 161 operates to Fourier transform the data into an array of image data. These image data are conveyed through the serial link 115 to the computer system 107 where they are stored in the disk memory 111. In response to commands received from the operator console 100, these image data may be archived on the tape drive 112, or they may be further processed by the image processor 106 and conveyed to the operator console 100 and presented on the display 104. Further processing is performed by the image processor 106 that includes reconstructing acquired MR image data. It is to be appreciated that a MRI scanner is designed to accomplish field homogeneity with given scanner requirements of openness, speed and cost.

In embodiments of the present invention, MR imaging is performed during an interventional procedure on subject 200 of FIG. 1 using for example an interventional device 210 and the MR scanner is configured to have multiple transmit and receive channels for acquiring MR signals. In a multiple channel configuration, the MR scanner is able to acquire multiple MR signals in order to locate interventional device 210, for example a catheter within the subject 200.

In accordance with embodiments of the present invention, a method for acquiring images of an interventional device 210 and its surroundings during MRI is provided. The method for acquiring images during an interventional procedure using a Magnetic Resonance Imaging (MRI) device comprises acquiring at least one planar image of a region of interest and acquiring at least one image of an interventional device projected onto the planar image substantially simultaneously. The method further comprises controlling at least one RF transmit channel in the MRI device in order to produce projection images of the interventional device during an imaging session.

In this embodiment, the method enables simultaneously achieving planar (e.g. two-dimension or 2D) imaging of the subject at any prescribed scan plane and projection imaging of a device/its surroundings, even in cases where the device is located entirely outside the prescribed scan plane. Key components of the method include integration of auxiliary transmit coils with the tracked device, control of the coils' RF transmission through additional RF transmit channels that operate in parallel with a scanner's principal RF transmit channel, and producing projection images in rapid succession.

In order to have a MRI method that enables simultaneous planar roadmap imaging and device tracking according to embodiments of the present invention, the MRI system of FIG. 1 employs multiple RF transmit channels (not shown) to enable simultaneity and to produce images showing device location as a projection onto the roadmap frames. The method may be advantageously applied in cases where soft tissue near the device undergoes motion/deformation and frequent roadmap image-update is desired.

For monitoring the placement of a catheter during an interventional procedure, a parallel RF transmit-based MRI imaging sequence through pulse generator 121 of FIG. 1 is employed. In an embodiment according to the present invention, the method integrates transmit coils with the catheter and controls the coils' RF transmission through additional channels that operate synchronously with a scanner's standard transmit channel. The method enables simultaneous planar roadmap imaging and device tracking, and automatically produces images showing device location as a projection onto the roadmap frames.

The method is applicable on an MR scanner that is equipped with multiple parallel RF transmit channels. The method involves integrating auxiliary RF transmit and/or receive coils with the tracked device(s), controlling the transmit and receive function of both the scanner's standard RF coil(s) and the auxiliary RF coils, and producing projection images in rapid succession.

In one embodiment, the MR imaging system of FIG. 1 further comprises at least one RF transmit channel (not shown) to drive the scanner's standard transmit coil (e.g., an originally installed whole-body birdcage coil) that is suitable for imaging the region of interest, and one or more additional RF transmit channels to drive corresponding auxiliary coils (to be described with reference to FIG. 3) that are integrated with the tracked device(s) 210. The driving of the regular transmit coil is done in such a way that it accomplishes slice-selective excitation of the region of interest. The driving of the auxiliary coils is done in such a way that it induces, simultaneous to the slice-selective excitation, non-selective or mildly-selective excitation that mostly excite spins near the auxiliary coils. A regular planar image acquisition sequence that acquires data from the standard receive coil(s) then leads to the formation of a projection image of the 3D magnetization distribution. This projection image, in a 2D format, captures both the selected slice and the surroundings of the auxiliary coils, the latter providing direct indication of the location of the tracked device(s). Repeated generation of such a projection image, with appropriate on-the-fly prescription of slice location, then allows dynamic visualization of both subject anatomy and device position.

In a further embodiment, the auxiliary coils are also configured to receive MR signal. The data collected by the auxiliary coils are processed and combined with the data from the standard receive coil(s) to achieve contrast enhancement of the projection 2D images.

The integration of auxiliary transmit coils with a tracked device can be done in many ways. To track a catheter for instance, one can attach small transmit coils, with or without water-filled vials, to a segment of the catheter at a set of selected locations, as shown in FIG. 2. Alternatively one can custom-make the catheter such that it additionally serves the function of RF transmit (i.e., a transmit catheter). In an exemplary embodiment, interventional device 10 includes at least one auxiliary transmit coil 12 coupled to at least one auxiliary transmit channel 14 of imaging system 1 (described above with reference to FIG. 1). Interventional device 10 is employed for insertion into region of interest 16.

In a further exemplary embodiment, a two-channel implementation is now described—while the MR scanner's principal RF transmit channel drives a regular or designated transmit coil (e.g., an originally installed whole-body birdcage coil) to accomplish slice selection, the second channel may drive an auxiliary transmit coil with a non-selective or mildly-selective narrow pulse that is played-out at a time instant chosen for obtaining proper gradient refocusing (see FIG. 3). With this implementation, both in-slice spins, which are excited by the regular slice selective pulse, and off-slice spins surrounding the auxiliary transmit coil, which are excited by the narrow pulse, contribute to the MR signal. A regular planar acquisition sequence may readily project the 3D magnetization distribution, creating a 2D image that shows the selected slice as well as the surroundings of the auxiliary coil. Referring to FIG. 3, (a) and (b) show components of a standard and well-known slice selection pulse sequence for obtaining the planar or 2D image of a region of interest. Pulse (c) shows a pulse sequence for use in driving the auxiliary transmit coil with a non-selective or mildly selective (hereinafter “substantially non-selective”) narrow pulse in order to induce spin excitation near the auxiliary transmit coils and obtain projections of the catheter onto the 2D image of the region of interest.

As part of the first embodiment, one can design the auxiliary coil(s) to change device-slice contrast and/or adjust the level of RF inputs to all transmit coils to alter device-slice contrast on the fly.

With this embodiment, superposition of device location information onto the roadmap planar image is automatically accomplished. And most significantly, the creation of such a “tracking+roadmap” image can be repeated as fast as regular planar roadmap imaging—acquiring and presenting tracking information does not incur speed penalty. With appropriate on-the-fly prescription of slice location, this facilitates high frame-rate visualization of both subject anatomy and device position.

Simultaneous roadmap imaging and device tracking were evaluated with the support of an 8-channel parallel transmit-capable MRI scanner and an adapted gradient echo sequence that accommodates the parallel excitation pulses shown in FIG. 3.

In terms of pulse sequence timing, parallel transmission of a narrow pulse on the second channel incurs no speed penalty. This implies fully retained freedom in optimizing the gradient echo sequence and good portability of the scheme to other rapid planar imaging sequences of choice.

In a further embodiment, the auxiliary coils are also configured to receive MR signal. The data collected by the auxiliary coils are processed and combined with the data from the standard receive coil(s) to achieve contrast enhancement of the projection 2D images. The data processing and combination may include, but not limited to, forming weighted sum of complex—valued images that are reconstructed from MR signal acquired from the multiple receive coils.

While the preferred embodiments of the present invention have been shown and described herein, it will be obvious that such embodiments are provided by way of example only. Numerous variations, changes and substitutions will occur to those of skill in the art without departing from the invention herein. Accordingly, it is intended that the invention be limited only by the spirit and scope of the appended claims.

What is claimed:

1. A method for acquiring images during an interventional procedure using a Magnetic Resonance Imaging (MRI) device comprising:
acquiring at least one planar image of a region of interest and acquiring at least one image of an interventional device projected onto the planar image substantially simultaneously.

2. The method of claim 1 wherein the image of an interventional device is obtained comprising the following steps:
integrating auxiliary transmit coils with the interventional device, wherein the auxiliary coils are coupled to at least one radiofrequency (RF) transmit channel of the MRI device; and,
controlling the at least one RF transmit channel in order to produce projection images of the interventional device during an imaging session.

3. The method of claim 1 wherein the MRI device is operated in a parallel transmit mode.

4. The method of claim 1 wherein the MRI device is further adapted to have multiple channels for transmitting and/or receiving radiofrequency (RF) signals.

5. The method of claim 2 further comprising the step of:
coupling signals from the at least one RF transmit channel to the auxiliary transmit coils of the interventional device and wherein the signals are used in controlling RF transmission of the auxiliary coils;
wherein the at least one RF transmit channel is in addition to and operating synchronously with a plurality of designated transmit channels of the MRI device.

6. The method of claim 5 wherein the designated transmit coils are responsive to a pulse sequence configured to accomplish slice-selective excitation of the region of interest.

7. The method of claim 6 wherein the auxiliary coils are responsive to a pulse sequence configured to induce, simultaneous to the slice-selective excitation of the designated transmit coils, a substantially non-selective excitation to excite spins near the auxiliary coils.

8. The method of claim 1 further comprising the step of generating and displaying a resulting projection image, in a two-dimensional (2D) format, wherein the resulting projection image captures both a selected slice and surroundings of the auxiliary coils, thereby providing an indication of a location of the interventional device.

9. The method of claim 8 further comprising repeating the step of generating and displaying the resulting projection image, with on-the-fly prescription of slice location, thereby allowing dynamic visualization of both the region of interest and the interventional device position.

10. The method of claim 1 wherein the interventional device further comprises at least one auxiliary tracking coil for at least one of transmitting or receiving radiofrequency (RF) signals to/from the MRI device.

11. A system for acquiring images during an interventional procedure using a multiple channel Magnetic Resonance Imaging (MRI) device comprising:
a plurality of designated channels integrated with the MRI device for transmitting and/or receiving radiofrequency (RF) signals during a MRI imaging session;
an interventional device comprising at least one auxiliary transmit coil; and,
a pulse sequence generator for generating pulse sequences adapted to acquire at least one planar image of a region of interest and at least one image of an interventional device projected onto the planar image substantially simultaneously.

12. The system of claim 11 wherein the interventional device comprises at least one auxiliary transmit coil coupled to at least one radiofrequency (RF) transmit channel of the MRI device and wherein the pulse sequence generator is adapted to control the at least one RF transmit channel in order to produce projection images of the interventional device during an imaging session.

13. The system of claim 11 wherein the MRI device is operated in a parallel transmit mode.

14. The system of claim 11 wherein the interventional device comprises at least one of a catheter, a probe, an invasive medical device and combinations thereof.

15. The system of claim 12 further comprising a processor configured for:
coupling signals from the at least one RF transmit channel to the auxiliary transmit coil of the interventional device and wherein the signals are used in controlling RF transmission of the auxiliary coils;
wherein the at least one RF transmit channel is in addition to and operating synchronously with a plurality of designated transmit channels of the MRI device.

16. The system of claim 15 wherein the designated transmit coils are responsive to a pulse sequence configured to accomplish slice-selective excitation of the region of interest.

17. The system of claim 16 wherein the auxiliary coils are responsive to a pulse sequence configured to induce, simultaneous to the slice-selective excitation of the designated transmit coils, a substantially non-selective excitation to excite spins near the auxiliary coils.

18. The system of claim 11 the further comprising a display for displaying a resulting projection image, in a two-dimensional (2D) format, wherein the resulting projection image captures both a selected slice and surroundings of the auxiliary coils, thereby providing an indication of a location of the interventional device.

19. The system of claim 18 wherein the display is configured for displaying the resulting projection image, with on-the-fly prescription of slice location, thereby allowing dynamic visualization of both the region of interest and the interventional device position.

20. The system of claim 11 wherein the interventional device further comprises at least one auxiliary tracking coil for at least one of transmitting or receiving radiofrequency (RF) signals to/from the MRI device.
